(19) United States
(12) Patent Application Publication
(10) Pub. No.: US 2009/0117522 A1
(43) Pub. Date: May 7, 2009

(54) System to whiten interproximal surfaces of adjacent teeth

(76) Inventor: Yaneav Cohen, Scottsdale, AZ (US)

Correspondence Address: TOD R NISSLE, PO BOX 55630, PHOENIX, AZ, 85078, US

(21) Appl. No.: 11/982,493

(22) Filed: Nov. 2, 2007

(51) Int. Cl. A61K 8/22

(52) U.S. Cl. 433/215; 433/216

(57) ABSTRACT

A method to whiten contacting interproximal mesial and distal surfaces of a pair of adjacent teeth includes the steps of providing a carrier shaped and dimensioned to be inserted between the pair of adjacent teeth, to fit between and separate the interproximal mesial and distal surfaces, and to contact each of the interproximal mesial and distal surfaces; charging the carrier with a tooth whitening composition; inserting the carrier intermediate the pair of adjacent teeth; and, manipulating the carrier to separate the contacting interproximal mesial and distal surfaces and to deliver the tooth whitening composition to the interproximal mesial and distal surfaces by contacting simultaneously the interproximal mesial and distal surfaces with said carrier to contact each of the interproximal mesial and distal surfaces with a portion of the tooth whitening composition on the carrier.

This invention relates to systems to whiten teeth.

A variety of tooth whitening systems are known. Such systems employ, for example, toothpaste, strips that are applied to teeth, and gels that are applied to teeth. While tooth whitening systems have long existed, there does not appear to be market pressure to change the design of current systems, nor does there appear to be any recognition of an overriding design need that would fuel a search for a change in design of such systems. The market trend, and the trend of the manufacturers of such systems, appears to be in favor of retaining and continuing to utilize existing tooth whitening systems. As a result, it is anticipated that most, if not all, prior patents or articles describing such prior art whitening systems will not discuss market pressure or a design need in connection with tooth whitening systems, or if such discussions are found in the prior patents or articles, they will comprise a minority of the teachings in the area or will concern design changes different from those set forth below.

In addition, the knowledge of a skilled artisan appears to comprise the prior art tooth whitening systems noted above. Consequently, there does not seem to be motivation for the artisan to consider significantly altering such apparatus, at least not in the manner set forth below.

However, as has been demonstrated many times in the Patent Office, existing apparatus and methods often can be improved by utilizing an unidentified, unanticipated combination which provides functions that are unpredictable in view of the prior art.

Accordingly, it would be highly desirable to provide an improved system to whiten teeth.

Therefore, it is a principal object of the invention to provide an improved system to whiten teeth.

A further object of the invention is to provide a system to whiten dental interproximal surfaces to include mesial and distal proximal contact surfaces of adjacent of teeth.

These and other, further and more specific objects and advantages of the invention are set forth below, in conjunction with the drawings, in which:

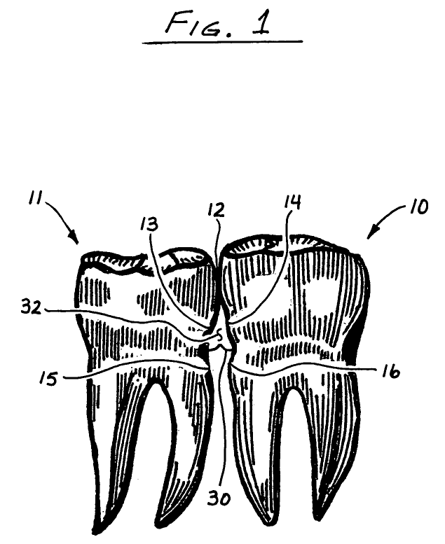

FIG. 1 is a perspective buccal view illustrating cervicoocclusally a pair of adjacent posterior teeth and cervicoocclusally the interproximal mesial and distal contact surfaces, the occlusal embrasure surface, and the interproximal space therebetween;

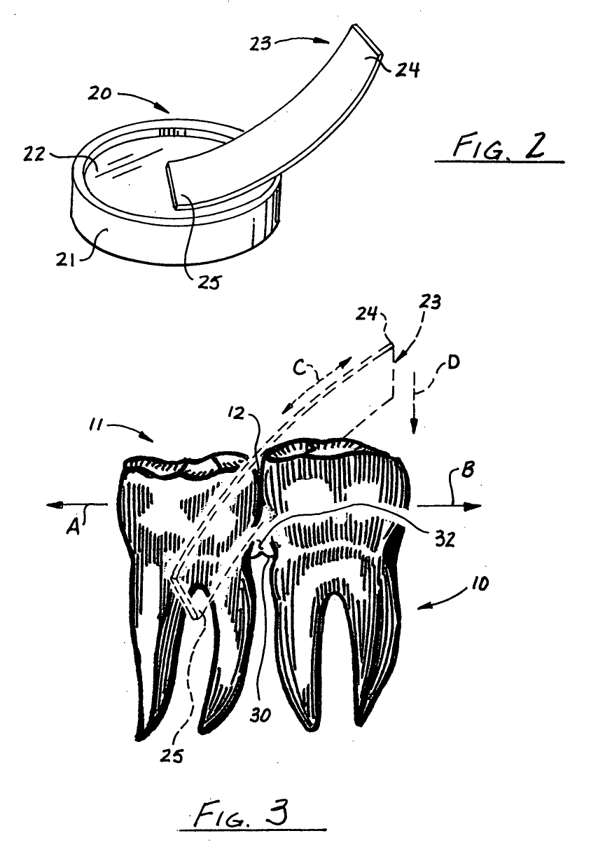

FIG. 2 is a perspective view illustrating the application of a whitening composition to a carrier; and,

FIG. 3 is a perspective view illustrating the use of the carrier of FIG. 2 to whiten interproximal contact areas to include mesial and distal proximal surfaces of adjacent teeth and the resulting embrasure surfaces therebetween.

Briefly, in accordance with my invention, I provide an improved method to whiten contacting interproximal mesial and distal surfaces of a pair of adjacent teeth. The method includes the steps of providing a carrier shaped and dimensioned to be inserted between the pair of adjacent teeth, to fit between and separate the contacting interproximal mesial and distal surfaces, and to contact each of the interproximal mesial and distal surfaces; charging the carrier with a tooth whitening composition; inserting the carrier intermediate the pair of adjacent teeth; and, manipulating the carrier to separate the contacting interproximal mesial and distal surfaces and to deliver the tooth whitening composition to the interproximal mesial and distal surfaces by contacting simultaneously the interproximal mesial and distal surfaces with the carrier to contact each of the interproximal mesial and distal surface with a portion of the tooth whitening composition on the carrier. An additional step can be included prior to inserting the carrier intermediate the adjacent teeth. The step comprises including the step intermediate steps (b) and (c) of determining the location of the contacting interproximal mesial and distal surface by examining the adjacent teeth from at least one aspect selected from a group consisting of the labial aspect, the buccal aspect, the lingual aspect, the incisal aspect, and the occlusal aspect.

Turning now to the drawings, which are presented by way of explanation and not limitation of the invention, and in which like reference characters refer to corresponding elements throughout the several views, FIG. 1 illustrates a pair of adjacent posterior teeth 10 and 11. Tooth 10 includes an interproximal mesial surface 14 that is spaced apart from and opposes interproximal distal surface 13 of tooth 11. The interproximal distal and mesial surfaces 13 and 14, respectively, normally extend above the gum line 30 of an individual, while opposing root surfaces 16 and 15 of teeth 10 and 11, respectively, extend below the gum line. For posterior teeth, buccal (not visible), lingual (not visible), and occlusal embrasures extend outwardly or upwardly, as the case may be, from the contact area 12 at which a mesial 14 and distal 13 interproximal surface contacts another tooth. When anterior teeth 10 and 11 are, as is the case in FIG. 1, viewed from the buccal aspect to determine the size of the occlusal embrasure and to determine the position of contact area 12 cervicoocclusally, area 12 is located further from the cervical line and closer to the occlusal surface of each tooth 10 and 11. For anterior teeth, the labial, lingual, and incisal embrasures extend outwardly or upwardly, as the case may be, from the area at which an anterior tooth contacts another tooth. In some instances, the occlusal or incisal embrasure can be relatively small.

In FIG. 2, a gum whitening composition 22 including carbamide peroxide, hydrogen peroxide, or any other desired tooth whitening composition or agent is stored in the base 21 of container 20. Container 20 normally also includes a cover (not shown) that is sealingly turned onto base 21 to seal composition 22 in container 20. The composition can comprise a liquid, a gel, a slurry, a powder, or any other desired type of composition. Prior to be inserted between a pair of adjacent teeth 11 and 12, the carrier or substrate 23 is contacted with composition 22 to charge the carrier 23 with composition 22. If the carrier 23 is permeable or porous with respect to composition 22, carrier 23 can be charged when the composition 22 is absorbed by carrier 23. If the carrier 23 is not permeable, the carrier 23 can be charged with the composition when the composition coats the exterior of the carrier. Or composition 22 can both coat and be absorbed by a carrier 23.

In one preferred embodiment of the invention, carrier 23 comprises a polymer strip and composition 22 is a gel or other composition which adheres to the surface of carrier 23. The carrier 23 is utilized between a pair of teeth 10, 11 to whiten an embrasure or an interproximal mesial and distal surface(s) 13 to 16 before the gel dries.

In another preferred embodiment of the invention, carrier 23 comprises a fabric strip and composition 22 is a liquid which is absorbed by, wets, and impregnates carrier 23. The carrier 23 is utilized between a pair of teeth 10, 11 to whiten an embrasure or an interproximal mesial or distal surface(s) 13, 14, 15, 16 before the impregnating liquid dries.

In a further preferred embodiment of the invention, carrier 23 comprises a pliable metal strip and the composition 22 is a liquid or gel which adheres to the surface of the strip and dries to form a dry layer including a whitening composition. The liquid or gel is allowed to dry on carrier 23 before the carrier is utilized between a pair of teeth 10, 11 to whiten an embrasure or an interproximal mesial or distal surface(s) 13 to 16.

As noted, the portion of the interproximal mesial and distal surfaces that is whitened in accordance with the invention can include contact area 12. The shape and dimension and structure of carrier 23 can vary as desired as long as carrier 23 can perform the functions of carrying a tooth whitening composition, of fitting between a pair of adjacent teeth 10 and 11, and of contacting an embrasure or an interproximal mesial or distal surface(s) 13 to 16. It is also preferred, although not necessary, that carrier 23 be able to perform the function of separating a pair of adjacent teeth 10 and 11 in the directions indicated by arrows A and B so that carrier 23 can move between and contact portions of embrasures and of interproximal mesial and distal surfaces 13 and 14 that normally contact each other in an area 12. In an alternate embodiment of the invention, carrier 23 is not utilized to separate a pair 10, 11 of adjacent teeth. Instead, a mechanical wedge is be inserted and forced between teeth 10, 11 to separate temporarily teeth 10 and 11 to expose portions of interproximal mesial and distal surfaces 13 to 16 that normally contact one another. After such portions of interproximal mesial and distal surfaces 13 to 16 are exposed, carrier 23 is placed intermediate teeth 10 and 11 and contacted with embrasures, with said exposed portions of interproximal mesial and distal surfaces 13 to 16, and with portions of interproximal mesial and distal surfaces that extend below the normal contact area 12. Orthodontic or other apparatus can be utilized to separate teeth 10, 11 to expose portions of a pair of opposing interproximal mesial 14 and distal 13 surfaces that normally contact one another. A carrier 23 functions to separate a pair of interproximal distal 13 and mesial 14 surfaces that contact at an area 12 when the carrier can be slid or otherwise moved or worked between teeth 10, 11 at area 12 such that carrier 23 displaces teeth 10 and 11 in the directions indicated by arrow B and A, respectively, (FIG. 3), and is intermediate distal surface 13 and mesial surface 14 at area 12 such that surfaces 13 and 14 do not contact each other at area 12 while carrier 23 is therebetween, and such that surfaces 13 and 14 instead contact carrier 23.

A carrier 23 charged with a tooth whitening composition 22 can be used to apply the whitening composition to a portion of an interproximal surface 13 to 16 that is at or slightly below the gum line of the tooth by simply contacting that portion of the interproximal surface.

The length of time that a carrier 23 is maintained in contact with an interproximal distal or mesial surface 13 to 16 can vary as desired, depending in part on the characteristics of composition 22. If, for example, composition 22 is fast acting, the time that carrier 23 is maintained in contact with a mesial or distal surface 13 to 16 may be relatively short. Or, if the composition 22 on a carrier 23 adheres to a surface 13 to 16 and functions over a period of time as a time release composition, the time that carrier 23 is maintained in contact with a mesial or distal surface 13 to 16 may be relatively short. Or, if the composition 22 acts slowly to whiten a distal or mesial surface 13 to 16, the carrier 23 may be maintained in contact with the surface 13 to 16 for a longer period of time.

A carrier 23 can be charged with a whitening composition 22 just prior to inserting the carrier 23 intermediate a pair of teeth; or, the carrier 23 can be charged with a composition 22 at any selected time prior to inserting carrier 23 intermediate a pair of teeth. The carrier 23 can, after being charged with a whitening composition 22, be stored in a container prior to be utilized intermediate a pair of teeth 10, 11 to whiten one or more interproximal distal and mesial surfaces 13 to 16 of the teeth.

In use, a carrier 23 is charged with a whitening composition 22. The carrier 23 is placed intermediate a pair of teeth 10, 11 in the manner indicated in FIG. 3 and in the occlusal embrasure (or incisal embrasure as the case may be). The carrier is then manipulated to contact selected interproximal mesial and distal surface(s) 13 to 16 with whitening composition 22. After carrier 23 contacts an interproximal mesial or distal surface, the carrier 23 can be held stationary against the surface, can be moved or slid in one direction over the surface, or can be moved back and forth over the surface. In FIG. 3, carrier 23 is manipulated by manually (or with a mechanical device) grasping ends 24 and 25 and moving carrier 23 in the directions of arrow C while pulling or pushing down on carrier in the direction of arrow D to move carrier 23 into contact with interproximal distal surface 13 and interproximal mesial surface 14 at area 12, to move carrier 23 between interproximal distal surface 13 and interproximal mesial surface 14 at the area 12 where the surface 13 and 14 normally contact one another, and, consequently, to interpose carrier 23 between and separate the normally contacting portions of interproximal distal surface 13 and interproximal mesial surface 14.

In the event that the occlusal or incisal embrasure is small, it may, in accordance with one embodiment of the invention, be advantageous to insert carrier 23 through the open interproximal space 32 that extends below contact area 12 and between spaced apart portions of distal surface 13 and mesial surface 14.

In one embodiment of the invention, the location of area 12 is ascertained before carrier 23 is utilized. This is accomplished by viewing area 12 from the incisal or occlusal aspect, as the case may be; and/or by viewing area from the labial or bucal aspect, as the case may be. When area 12, and the adjacent anterior or posterior teeth with contacting mesial and distal surfaces in area 12, is viewed from the incisal or occlusal aspect, the lateral position of area 12 is adjudged labiolingually or buccolingually, as the case may be. When area 12, and the adjacent anterior or posterior teeth with contacting mesial and distal surfaces in area 12, is viewed from the labial or buccal aspect, the vertical position of area 12 is adjudged cervicoincisally or cervicoocclusally, as the case may. If area 12 is, after examining area 12 from an incisal or occlusal aspect as the case may be, seen to be closer to the labial or buccal side of the teeth, it may be advantageous to insert initially carrier 23 between adjacent teeth and into area 12 from the labial or buccal side of the adjacent pair of teeth. If area 12 is, after examining area 12 from the incisal or occlusal aspect as the case may be, seen to be closer to the lingual side of the teeth, it may be advantageous to insert initially carrier 23 between adjacent teeth and into area 12 from the lingual side of the adjacent pair of teeth. If area 12 is, after examining area 12 from a labial or buccal aspect as the case may be, seen to be closer to the gum line than to the occlusal plane of the teeth, it may be advantageous to insert initially carrier 23 between adjacent teeth and into area 12 at a point nearer the gum line. If area 12 is, after examining area 12 from a labial or buccal aspect as the case may be, seen to be closer to the occlusal plane of the teeth than to the gum line, it may be advantageous to insert initially carrier 23 between adjacent teeth and into area 12 at a point nearer the occlusal plane. In sum, an important feature in this embodiment of the invention is examining area 12 from the incisal or occlusal aspect, as the case may be, and/or from the labial or bucal aspect, as the case may be, to ascertain the location of area 12 toward in the inside or outside of a pair of adjacent teeth and/or toward the gum line or occlusal plane of the teeth. Such knowledge of the location of area 12 can be useful in determining how to initially insert carrier 23 between a pair of adjacent teeth and into area 12. In one instance it may be desirable to insert initially a carrier 23 between a pair of adjacent teeth at a location spaced further away from area 12, this for carrier 23 to gain position between the adjacent teeth prior to being moved into area 12. In another instance it may be desirable to insert initially a carrier 23 at or as close as possible to area 12.

Having described my invention in such terms as to enable those of skill in the art to understand and practice it, and having described the presently preferred embodiments thereof, I claim:

1. A method to whiten contacting interproximal mesial and distal surfaces of a pair of adjacent teeth, comprising the steps of
(a) providing a carrier shaped and dimensioned to
(i) be inserted between the pair of adjacent teeth,
(ii) fit between and separate the interproximal mesial and distal surfaces,
(iii) contact each of the interproximal mesial and distal surfaces;
(b) charging said carrier with a tooth whitening composition;
(c) inserting said carrier intermediate the pair of adjacent teeth;
(d) manipulating the carrier to separate the contacting interproximal mesial and distal surfaces and to deliver said tooth whitening composition to the interproximal mesial and distal surfaces by contacting simultaneously the interproximal mesial and distal surfaces with said carrier to contact each of the interproximal mesial and distal surfaces with a portion of the tooth whitening composition on the carrier.

2. The method of claim 1 including the step intermediate steps (b) and (c) of determining the location the contacting interproximal mesial and distal surfaces by examining the adjacent teeth from at least one aspect selected from a group consisting of the labial aspect, the buccal aspect, the lingual aspect, the incisal aspect, and the occlusal aspect.
